(19) United States
(12) Patent Application Publication
(10) Pub. No.: US 2025/0249277 A1
(43) Pub. Date: Aug. 7, 2025

(54) LIGHT OUTPUT DEVICE FOR SCALP CARE INCLUDING THIN FILM-TYPE HEAT DISSIPATION SHEET

(71) Applicant: LG ELECTRONICS INC., Seoul (KR)

(72) Inventors: Eunjin OH, Seoul (KR); Hyeuk CHANG, Seoul (KR); Hyoungjun KIM, Seoul (KR)

(73) Assignee: LG ELECTRONICS INC., Seoul (KR)

(21) Appl. No.: 19/011,282

(22) Filed: Jan. 6, 2025

(30) Foreign Application Priority Data
Feb. 7, 2024 (KR) 10-2024-0018521
Nov. 29, 2024 (WO) PCT/KR2024/019302

(51) Int. Cl. A61N 5/06; A61N 5/00; A61N 5/067

(52) U.S. Cl. CPC A61N 5/0617; A61N 5/067; A61N 2005/005; A61N 2005/0647; A61N 2005/0652; A61N 2005/0659; A61N 2005/0663

(57) ABSTRACT

The present disclosure relates to a light output device for scalp care including a thin film-type heat dissipation sheet. The heat dissipation sheet is composed of an insulating layer and a heat diffusion layer to effectively block and disperse heat generated inside the device. This may protect user's skin from the heat of the device and prevent overheating of internal components, thereby improving durability and stability of the device.

### CROSS-REFERENCE TO RELATED APPLICATION(S)

Pursuant to 35 U.S.C. § 119, this application claims the benefit of earlier filing date and right of priority to Korean Application No. 10-2024-0018521, filed on Feb. 7, 2024 and International Application No. PCT/KR2024/019302, filed on Nov. 29, 2024, the contents of which are all incorporated by reference herein in their entirety.

### BACKGROUND

### Field

The present disclosure relates to a light output device, and more specifically, to a light output device for scalp care including a thin film-type heat dissipation sheet.

### Discussion of the Related Art

Modern people recognize their appearance as an important factor in showing and expressing themselves to others, and accordingly, they invest a lot of money and time in appearance management to overcome their flaws in the appearance.

Among aspects of the appearance, hair loss occurs because of environmental causes, genetic causes, mental causes such as stress, and physical causes such as hormonal imbalances. Such hair loss has mainly occurred in middle-aged and older people, but recently, not only has the hair loss increased among young men in their 20s and 30s, but young women are also exposed to the hair loss because of malnutrition caused by excessive dieting, frequent perms, dyeing, and the like. In line with this trend, various scalp care devices for preventing or treating the hair loss have been developed recently.

Low level laser therapy (LLLT) is a therapy that irradiates a light beam of a specific wavelength to biological tissue to promote metabolism in a relevant area and activate a function of the tissue. Laser light used in the low level laser therapy penetrates the biological tissue and activates ions in cells, thereby increasing production of capillaries, increasing oxygen concentration in blood, and promoting collagen production.

Based on such low level laser therapy, methods and devices that promote hair growth by irradiating the laser light to scalp to activate hair follicles and biological tissues around the hair follicles are emerging.

However, in the case of devices that irradiate the laser light to the scalp as described above, a plurality of light sources (such as LEDs) are disposed to output light toward user's skin.

When heat generated from the light source directly touches the skin or is transmitted at a short distance, it may cause excessive temperature rise in the skin, which may damage a skin barrier. In particular, when the skin is exposed to the heat for a long time, there is a risk of local inflammation or burns caused by the heat, so that the user is likely to experience continuous discomfort.

However, in an environment with a narrow internal space like the scalp care device, it is difficult to apply an existing insulating material, so that it is not available to effectively block and disperse the heat. Therefore, a structure that minimizes direct heat contact with the skin and effectively disperses the heat is needed.

### SUMMARY

To solve the above problems, the present disclosure is intended to provide a thin film-type heat dissipation sheet that may be applied even in a very narrow space.

In addition, the present disclosure is intended to provide a safe scalp care environment by applying a heat dissipation sheet that may effectively insulate and diffuse heat to a scalp care device.

The problems to be solved in the present disclosure may not be limited to the technical problems mentioned above, and other technical problems not mentioned may be clearly understood by a person having ordinary skill in the technical field to which the present disclosure belongs from the description below.

A light output device for scalp care includes a dome-shaped outer casing forming an outer appearance of the device, an inner casing formed inside the outer casing, a plurality of light sources arranged in a space between the outer casing and the inner casing, a controller disposed in the space between the outer casing and the inner casing, and a thin film-type heat dissipation sheet positioned adjacent to the controller, and the thin film-type heat dissipation sheet includes an insulating layer made of a flexible material including an aerogel, and a heat diffusion layer formed on one surface of the insulating layer.

The controller may be disposed in a rear side of the space between the outer casing and the inner casing, and the thin film-type heat dissipation sheet may be disposed in a space between the controller and the inner casing.

The thin film-type heat dissipation sheet may further include a coating layer attached to a remaining surface of the insulating layer or one surface of the heat diffusion layer.

The coating layer may be formed on all of the remaining surface of the insulating layer, the one surface of the heat diffusion layer, a side surface of the insulating layer, and a side surface of the heat diffusion layer to surround the insulating layer and the heat diffusion layer.

The heat diffusion layer may be formed on both surfaces of the insulating layer.

The insulating layer may include glass fibers and silica gel particles having a diameter in a range of 2 to 50 nm.

A ratio of the glass fibers and the silica gel particles may be substantially 7 to 3.

The heat diffusion layer may be made of expanded graphite.

A thickness of the insulating layer may be equal to or smaller than 0.5 mm.

A thickness of the insulating layer may be equal to or smaller than 0.4 mm.

The thin film-type heat dissipation sheet may have a total cross-sectional thickness equal to or smaller than 1 mm.

According to at least one embodiment of the present disclosure, the effective heat management may be achieved even within the narrow device using the composite of the aerogel insulating sheet and the heat diffusion sheet.

In addition, according to at least one embodiment of the present disclosure, the performance degradation resulted from the overheating of the electronic component may be prevented and long-term stable operation may be enabled by maintaining the temperature inside the device uniformly via the insulating sheet and the heat dissipation sheet.

In addition, according to at least one embodiment of the present disclosure, risk of skin irritation and burns may be reduced by effectively blocking or dispersing the heat generated from the device to minimize the direct heat contact with the skin.

Further scope of the applicability of the present disclosure will become apparent from the detailed description below. However, because various changes and modifications within the spirit and scope of the present disclosure will be apparent to those skilled in the art, it should be understood that the detailed description and specific embodiments, such as preferred embodiments of the present disclosure, are given by way of example only.

### BRIEF DESCRIPTION OF THE DRAWINGS

FIG. 1 is a perspective view of a light output device for scalp care according to an embodiment of the present disclosure.

FIG. 2 is an exploded perspective view of a care body of a light output device shown in FIG. 1 as viewed from above.

FIG. 3 is an exploded perspective view of a care body of a light output device shown in FIG. 1 as viewed from below.

FIG. 4 is a view for more specifically illustrating a support included in a care body.

FIG. 5 is a bottom view of a care body light source mounting portion included in a care body.

FIG. 6 is a bottom view of a care body of a light output device illustrated in FIG. 1.

FIG. 7 is a view for illustrating a care body of the present disclosure having an arrangement form of light sources based on a hair loss type.

FIG. 8 is a view for illustrating a location of a thin film-type heat dissipation sheet according to an embodiment of the present disclosure.

FIG. 9 is a cross-sectional schematic diagram of a thin film-type heat dissipation sheet according to an embodiment of the present disclosure.

FIG. 10 is a cross-sectional schematic diagram of a thin film-type heat dissipation sheet according to another embodiment of the present disclosure.

FIG. 11 is a block diagram showing control components of a light output device for scalp care according to an embodiment of the present disclosure.

### DESCRIPTION OF SPECIFIC EMBODIMENTS

Description will now be given in detail according to exemplary embodiments disclosed herein, with reference to the accompanying drawings. The same or equivalent components may be provided with the same reference numbers, and description thereof will not be repeated. As used herein, the suffixes “module” and “part” are added or used interchangeably to facilitate preparation of this specification and are not intended to suggest distinct meanings or functions. In describing embodiments disclosed in this specification, relevant well-known technologies may not be described in detail in order not to obscure the subject matter of the embodiments disclosed in this specification. In addition, it should be noted that the accompanying drawings are only for easy understanding of the embodiments disclosed in the present specification, and should not be construed as limiting the technical spirit disclosed in the present specification. As such, the present disclosure should be construed to extend to any alterations, equivalents and substitutes in addition to those which are particularly set out in the accompanying drawings.

Although the terms first, second, etc. may be used herein to describe various elements, these elements should not be limited by these terms. These terms are generally only used to distinguish one element from another.

It will be understood that when an element is referred to as being “connected with” another element, the element can be directly connected with the other element or intervening elements may also be present. In contrast, it will be understood that when an element is referred to as being “directly connected with” another element, there are no intervening elements present.

A singular representation may include a plural representation unless it represents a definitely different meaning from the context.

The terms such as “include” or “have” used herein are intended to indicate that features, numbers, steps, operations, elements, components, or combinations thereof used in the following description exist and it should be thus understood that the possibility of existence or addition of one or more different features, numbers, steps, operations, elements, components, or combinations thereof is not excluded.

Hereinafter, embodiments of the present disclosure will be described in detail with reference to the drawings attached to the present document.

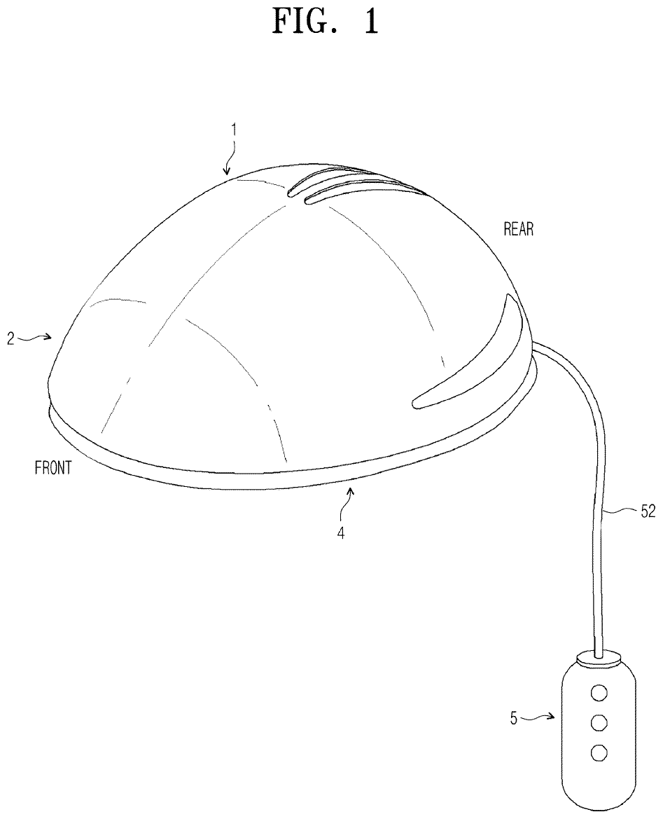

FIG. 1 is a perspective view of a light output device for scalp care according to an embodiment of the present disclosure.

Referring to FIG. 1, a light output device 1 for scalp care (hereinafter, referred to as a “light output device”) according to an embodiment of the present disclosure may be implemented to be worn on a head of a user and output light to user's scalp. Such light output device 1 may provide a function of promoting hair growth via activation of hair follicle cells, increase in production of capillaries, increase in oxygen concentration in blood, promotion of collagen production, and the like as light is output.

Such light output device 1 may include a care body 2, a circumference adjustor 4, and a manipulation device 5.

The care body 2 may be formed in a kind of dome shape to correspond to a shape of a human head, so that light may be evenly irradiated to the user's scalp.

Depending on the embodiment, the care body 2 may be formed to have a greater curvature at a front portion than at a rear portion when viewed from above in terms of similarity to the shape of the human head, but this may not be necessarily the case.

That is, in the present document, the term “dome” refers to a concept that includes not only a geometric dome itself but also a shape similar to the dome. In the present document, the shape similar to the dome may mean a shape having an arch shape (or a streamline shape) in each of a left and right direction and a front and rear direction.

The care body 2 may be equipped with a plurality of light sources that output light for scalp care of the user. The plurality of light sources may include laser light sources (e.g., laser diodes) that output laser light and LEDs. For example, the plurality of light sources may emit red light having a wavelength of about 630 nm to 670 nm, but may emit red light or infrared light without being limited thereto. Red light may promote the hair growth via the activation of the hair follicle cells or the like.

In one example, the laser light emitted by the laser light source may have an intensity greater than that of light emitted from the LED and may penetrate deeper into skin, thereby providing a higher scalp care effect. Accordingly, the laser light source in the care body 2 is positioned to irradiate the laser light to an area where the scalp care is more necessary when the user wears the device, thereby enabling intensive care for the area. A care body light source unit disposed in the care body 2 will be described in more detail later with the drawings.

In one example, the care body 2 may further include various sensors, such as a sensor for measuring a user's scalp condition (a temperature, a humidity, and the like), at least one sensor for sensing a user's hair loss type, and a sensor for sensing whether the light output device is worn.

The circumference adjustor 4 may be formed at a lower end of the care body 2 and may come into contact with a head circumferential surface when the user wears the device. The circumference adjustor 4 may include a structure whose length may be adjusted to correspond to head circumferential surfaces of various users. By the circumference adjustor 4, the care body 2 may be stably worn on the user's head and irradiate light to the scalp.

In one example, the light output device 1 may further include the user manipulation device 5 connected to the care body 2. For example, the user manipulation device 5 may be connected to the care body 2 by wire via a cable 52 or the like, but may be connected thereto wirelessly via a wireless communication scheme without being limited thereto.

For example, the user manipulation device 5 may be formed in a cylindrical shape such that the user may easily hold and use the same by hand. The user manipulation device 5 may have at least one button as an input unit for manipulating the light output device 1.

The user manipulation device 5 may provide an interface for the user to turn on/off the care body 2 or set an operation mode of the care body 2. Because the user manipulation device 5 is implemented as a component separate from the care body 2, the user may conveniently adjust an operation of the care body 2 using the user manipulation device 5 even while wearing the care body 2.

In addition, the user manipulation device 5 may be equipped with a battery that provides power for an operation of the light output device. Because the battery is equipped in the user manipulation device 5, a weight of the care body 2 may be minimized, thereby minimizing user inconvenience. Control components included in the user manipulation device 5 will be described later with reference to FIG. 11.

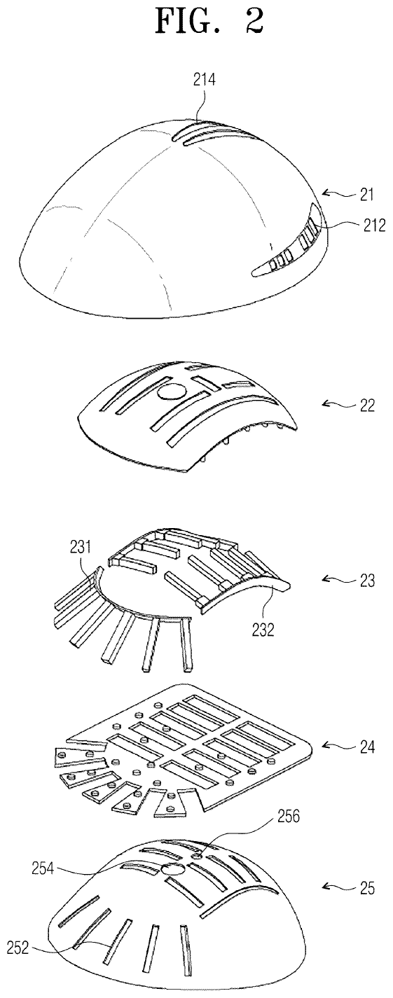

Hereinafter, embodiments related to a structure of the care body 2 and an arrangement of the light sources will be described in more detail with reference to FIGS. 2 to 7.

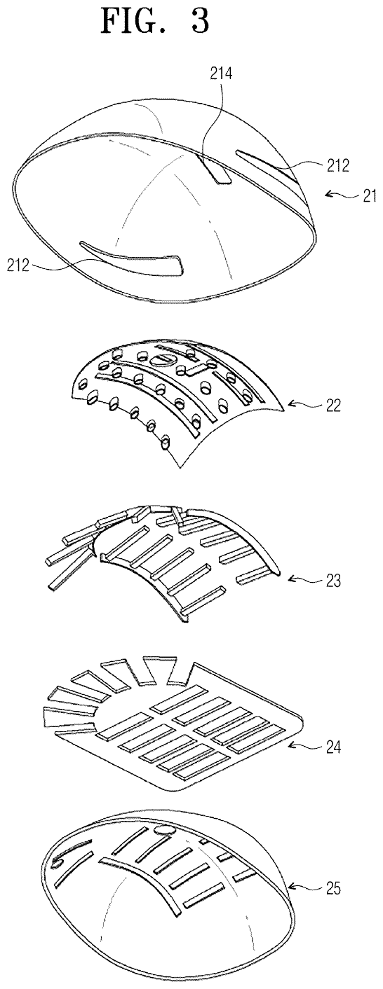

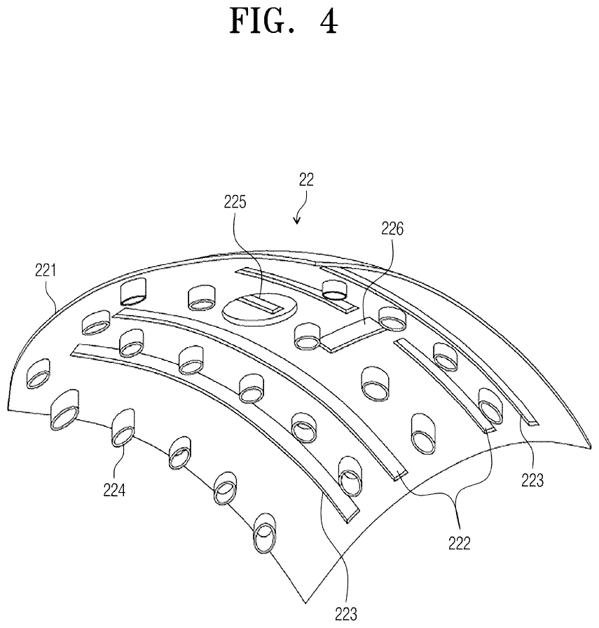

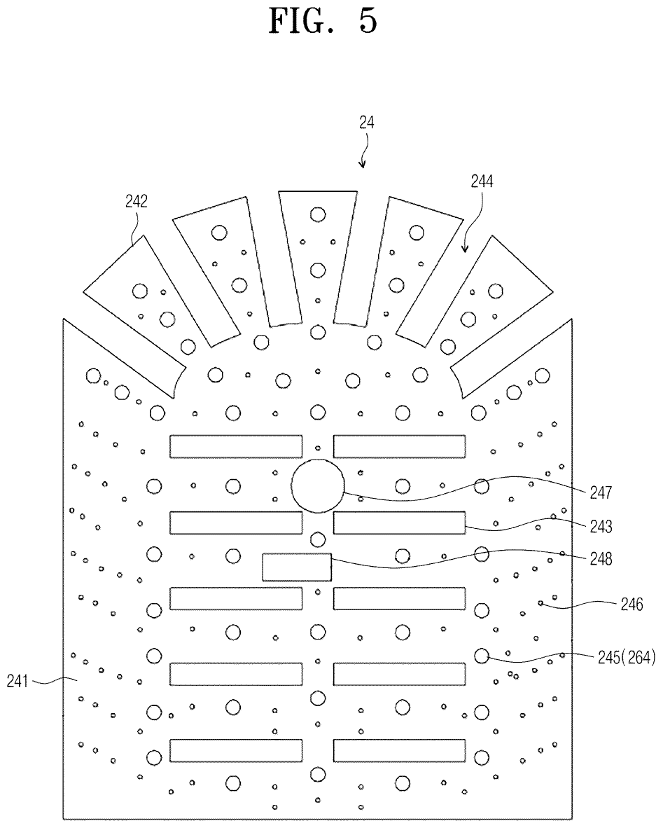

FIG. 2 is an exploded perspective view of a care body of a light output device shown in FIG. 1 as viewed from above. FIG. 3 is an exploded perspective view of a care body of a light output device shown in FIG. 1 as viewed from below. FIG. 4 is a view for more specifically illustrating a support included in a care body. FIG. 5 is a bottom view of a care body light source mounting portion included in a care body.

Referring to FIGS. 2 and 3, the care body 2 may include an outer casing 21, a support 22, a light guide 23, a care body light source mounting portion 24, and an inner casing 25.

The outer casing 21 may form an overall outer appearance of the care body 2. For example, the outer casing 21 may be made of a material such as plastic or stainless steel (SUS), and may protect internal components of the care body 2 from the outside. In one example, the outer casing 21 may be formed opaque, and may block light emitted from the laser light sources or the LEDs disposed inside from being irradiated to the outside.

According to embodiment, the outer casing 21 may have a lateral opening 212 and/or an upper opening 214 defined therein. When the care body 2 is worn, air may circulate between inside of the care body 2 (a space between the care body 2 and the head) and outside of the care body 2 via the lateral opening 212 and/or the upper opening 214. Accordingly, heat generated by operation of the light sources of the care body 2 may be effectively released to the outside, thereby preventing performance degradation of the light sources.

Referring to FIGS. 2 to 4, the support 22 may be disposed under the outer casing 21.

A support main body 221 forming an overall outer appearance of the support 22 may be formed in an arch shape in each of the left and right direction and the front and rear direction corresponding to the shape of the outer casing 21.

The support 22 may be fastened to an inner surface of the outer casing 21, but this may not be necessarily the case. For example, when the support 22 is implemented to be fastened to the inner surface of the outer casing 21, at least one fastening groove 222 may be defined in the support body 221, and at least one fastening protrusion corresponding to the at least one fastening groove 222 may be formed on the inner surface of the outer casing 21. In one example, the at least one fastening groove 222 may function as an opening that enables air circulation between the inside and the outside of the care body 2.

In one example, the support 22 may support and fix the light guide 23 and the care body light source mounting portion 24 relative to the outer casing 21 and/or the inner casing 25.

Referring to FIG. 4, at least one light guide fastening groove 223 may be defined in the support main body 221. For example, the at least one light guide fastening groove 223 may be formed in a shape of an opening in a partial area of the support main body 221. The light guide 23 may be supported and fixed by the support 22 as a laser light source mounting portion 232 of the light guide 23 is inserted into the light guide fastening groove 223.

In addition, a plurality of PCB fixing portions 224 may be formed on a bottom surface of the support main body 221.

As an example, each of the plurality of PCB fixing portions 224 may define an accommodation space into which each of a plurality of protrusions-to-be-inserted formed on a top surface of the care body light source mounting portion 24 is inserted and accommodated. When the plurality of protrusions-to-be-inserted are formed in a circular or cylindrical shape, the PCB fixing portions 224 may be implemented as protrusions having a circular ring shape as illustrated in FIG. 4. In this case, an outer diameter of each of the plurality of protrusions-to-be-inserted may be equal to or smaller than an inner diameter of each of the PCB fixing portions 224, so that the plurality of protrusions-to-be-inserted may be accommodated in and fixed in the accommodation space. The embodiment is for convenience of description. The shape of the plurality of PCB fixing portions 224 may not be limited thereto, and the plurality of PCB fixing portions 224 may be implemented in various shapes for fixing and supporting the care body light source mounting portion 24.

Depending on the embodiment, the support main body 221 may further be formed with a temperature/humidity sensor fixing portion 225 and an image sensor fixing portion 226. The temperature/humidity sensor fixing portion 225 may be formed to correspond to an upper portion of a temperature/humidity sensor mounted on the care body light source mounting portion 24 to fix and support the temperature/humidity sensor. The image sensor fixing portion 226 may be implemented as an opening through which an upper portion of the image sensor mounted on the care body light source mounting portion 24 passes to be fixed and supported.

That is, the care body light source mounting portion 24 may be fixed to and supported by the support 22 by the PCB fixing portions 224, the temperature/humidity sensor fixing portion 225, and the image sensor fixing portion 226. In one example, depending on the embodiment, the support 22 may include a fastening portion that is directly fastened to the care body light source mounting portion 24 to fix and support the care body light source mounting portion 24.

With continued reference to FIGS. 2 and 3, the light guide 23 and the care body light source mounting portion 24 may be disposed between the support 22 and the inner casing 25.

The light guide 23 may include a plurality of light guide mechanisms 231, and the laser light source mounting portion 232 including a first laser light source corresponding to each of the plurality of light guide mechanisms 231.

The plurality of light guide mechanisms 231 may distribute and irradiate laser light emitted from the respective first laser light sources corresponding thereto to a plurality of areas. The first laser light source may be disposed so as not to directly irradiate laser light in a user's head direction (or an inner casing direction).

The light guide mechanism 231 may be formed in a rod shape, and the first laser light source may emit the laser light in a longitudinal direction of the light guide mechanism 231.

In particular, the light guide mechanisms 231 may be disposed at an angle closer to a tangent line than to a vertical line to the tangent line at a location corresponding to the inner casing 25 in the longitudinal direction. For example, the light guide mechanism 231 may be disposed such that the longitudinal direction thereof is parallel to the tangent line, but this may not be necessarily the case.

Such light guide mechanism 231 may reflect the laser light emitted from the first laser light source and irradiate the light in the user's head direction (or the inner casing direction).

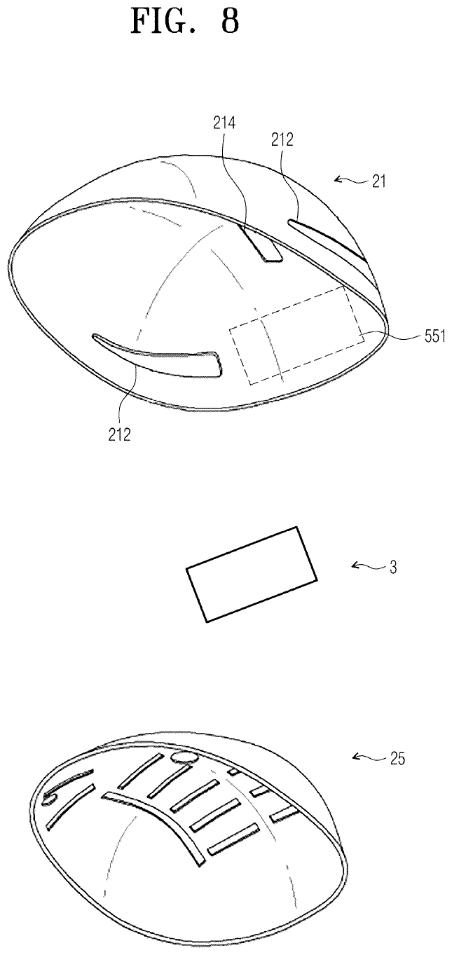

Specific details of the light guide mechanisms 231 will be described in more detail later with reference to FIGS. 8 to 12.

The laser light source mounting portion 232 may include a plurality of PCBs, each of which is equipped with at least one first laser light source. Although the laser light source mounting portion 232 including three PCBs is illustrated in FIGS. 2 to 3, the number of PCBs is not limited thereto. In one example, the PCB may be implemented as a flexible PCB (FPCB) having flexibility.

The light guide mechanisms 231 may be fastened to the laser light source mounting portion 232. In particular, each light guide mechanism 231 may be fastened to correspond to a location of each first laser light source mounted on the laser light source mounting portion 232.

As the laser light source mounting portion 232 is inserted into the light guide fastening groove 223 of the support 22, the light guide 23 may be fixed to and supported by the support 22.

A plurality of second laser light sources (laser diodes) and a plurality of LEDs may be mounted on the care body light source mounting portion 24, and a circuit pattern for supplying power to the plurality of second laser light sources and the plurality of LEDs may be formed. For example, the plurality of second laser light sources and the plurality of LEDs may be disposed to irradiate light downward of the care body light source mounting portion 24 (in the inner casing 25 direction).

In this regard, referring to FIG. 5, the care body light source mounting portion 24 may include a substrate 241. The substrate 241 may be implemented as a FPCB with flexibility. Accordingly, the care body light source mounting portion 24 may be bent into a dome shape (or an arch shape) corresponding to the shapes of the outer casing 21 and the inner casing 25. The care body light source mounting portion 24 may be fixed and supported by the support 22 described above, so that the bent state thereof may be stably maintained.

In one example, the substrate 241 may include a plurality of openings 243 defined at locations respectively corresponding to some of the plurality of light guide mechanisms 231. An area size of the opening 243 may be equal to or greater than an area size of the light guide mechanism 231. The light guide mechanism 231 may be disposed on the opening 243 corresponding thereto, or may be disposed in a lower portion of the substrate 241, with a portion thereof including a bottom surface extending through the opening 243.

Accordingly, the laser light emitted from each of the plurality of light guide mechanisms 231 may be irradiated to the user's scalp via the openings 243 defined at the locations corresponding thereto.

In one example, a plurality of branch substrates 242 may be formed at one edge of the substrate 241. The plurality of branch substrates 242 may extend from the one edge of the substrate 241. In FIG. 5, an example in which the plurality of branch substrates 242 extend radially from the one edge of the substrate 241 is illustrated, but this is not necessarily the case.

For example, the substrate 241 may have three edges formed in a square shape, and a remaining one edge may include two straight sections and a curved section formed between the two straight sections. The curved section may be formed convexly in an outward direction of the substrate 241. Each of the plurality of branch substrates 242 may extend from the curved section.

The plurality of branch substrates 242 may be formed to be spaced apart from each other by a predetermined distance. In addition, the branch substrates 242 located at the edge may be formed to be also spaced apart from the one edge (e.g., the straight section) of the substrate by a predetermined distance. Accordingly, a plurality of gap areas 244 may be defined between the plurality of branch substrates 242 and between the branch substrate 242 located at an edge and the substrate 241. The plurality of gap areas 244 may be defined to correspond to the light guide mechanisms 231 that are relatively forwardly positioned among the plurality of light guide mechanisms 231. The light guide mechanism 231 may be disposed on the gap area 244 corresponding thereto, or may be disposed in the lower portion of the substrate 241, with a portion thereof including the bottom surface extending through the gap area 244.

Accordingly, the laser light emitted from each of the light guide mechanisms 231 arranged in the front may be irradiated to the user's scalp via the gap areas 244.

In one example, a plurality of second laser light sources 245 and LEDs 246 may be arranged to be spaced apart from each other on a bottom surface of the substrate 241.

Output of the second laser light source 245 may be lower than output of a first laser light source 1000 (see FIG. 11) disposed on the light guide 23, but this may not be necessarily the case. In addition, output of the LEDs 246 may be lower than output of each of the first laser light source 1000 and the second laser light source 245.

In one example, the number of first laser light sources 1000 may be smaller than the number of second laser light sources 245, and the number of second laser light sources 245 may be smaller than the number of LEDs 246.

The laser light sources 1000 and 245 and the LEDs 246 may output red light. For example, the red light may have a wavelength of about 630 nm to 670 nm, but this may not be necessarily the case. Depending on the embodiment, the laser light sources 1000 and 245 and the LEDs 246 may output infrared light having a wavelength of about 780 nm to 1 mm.

In one example, each of the second laser light sources 245 may be equipped with a photodiode 264 that senses an amount of light. The light output device 1 may accurately sense a user's hair loss condition, hair loss type, or the like using an image sensor 262 and the plurality of photodiodes 264.

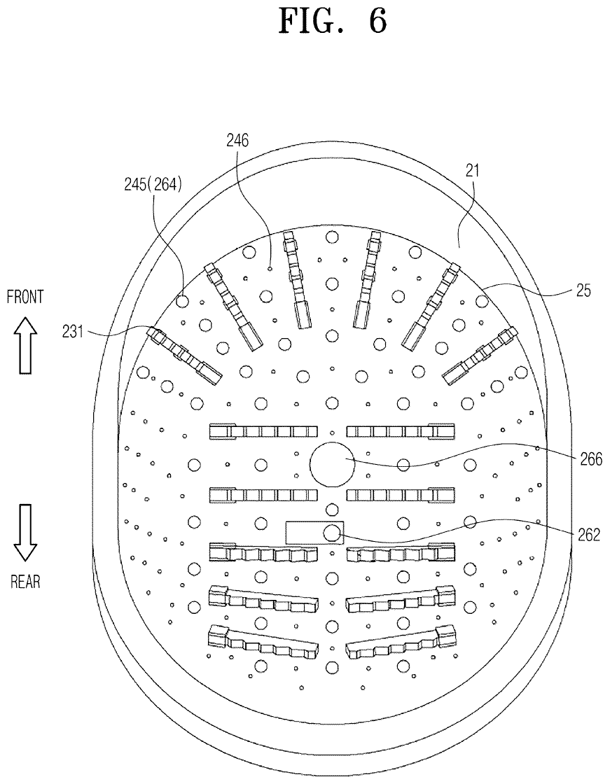

In addition, a temperature/humidity sensor mounting area 247 in which a temperature/humidity sensor 266 (see FIG. 6) is mounted and an image sensor mounting area 248 in which the image sensor 262 (see FIG. 6) is mounted may be formed on the substrate 241. For example, each of the temperature/humidity sensor mounting area 247 and the image sensor mounting area 248 may be formed closer to a center than to the edge of the substrate 241 and thus may be positioned on a user's parietal area (or a crown of the head) when the care body 2 is worn. Accordingly, the temperature/humidity sensor 266 may effectively sense heat or moisture generated from the user's head. In addition, the image sensor 262 may effectively obtain an image for sensing whether there is hair loss on the parietal area or the crown of the head.

In one example, a care body light source driver operating the plurality of laser light sources and the plurality of LEDs, and a sensor controller controlling the image sensor 262, the temperature/humidity sensor 266, and the like may be implemented on a separate PCB disposed inside or outside the care body 2. Depending on the embodiment, the care body light source driver and the sensor controller may be implemented on the care body light source mounting portion 24.

Referring to FIGS. 2 and 3, the inner casing 25 may be formed at an innermost side of the care body 2. The support 22, the light guide 23, and the care body light source mounting portion 24 described above may be accommodated between the outer casing 21 and the inner casing 25 and protected from the outside.

The inner casing 25 may have a dome shape corresponding to the shape of the outer casing 21. The inner casing 25 may be smaller in size than the outer casing 21, but this may not be necessarily the case.

The inner casing 25 may be made of a material such as transparent plastic or silicone, so that the light emitted from the laser light sources and the LEDs accommodated inside may be irradiated to the user's scalp through the inner casing 25.

Depending on the embodiment, a plurality of light guide mechanism openings 252 corresponding to the locations of the plurality of light guide mechanisms 23 may be defined in the inner casing 25. The laser light emitted from the light guide 23 may be irradiated to the user's scalp via the plurality of light guide mechanism openings 252.

In addition, to improve sensing accuracy of the temperature/humidity sensor 266 and the image sensor 262, a temperature/humidity sensor opening 254 corresponding to the temperature/humidity sensor 266 and an image sensor opening 256 corresponding to the image sensor 262 may be further defined in the inner casing 25.

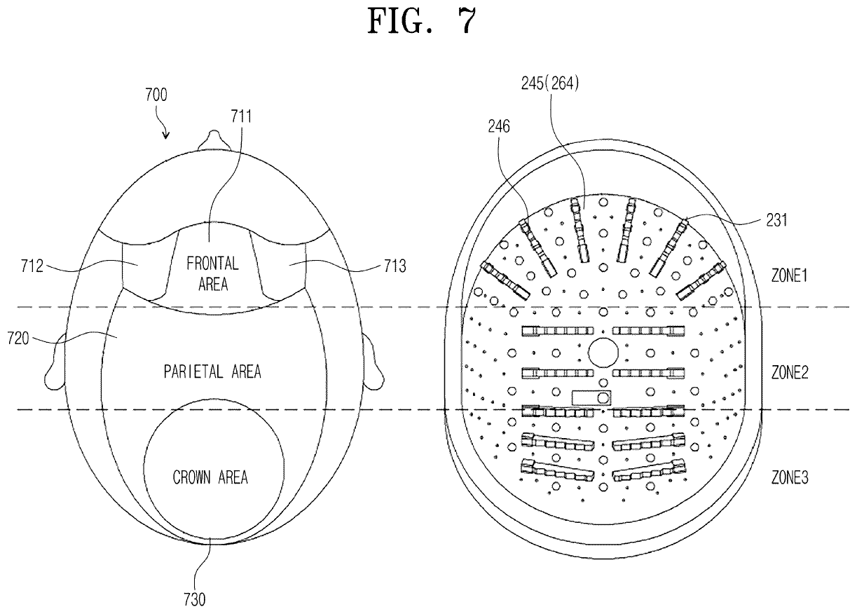

Hereinafter, characteristics related to the arrangement of the light sources of the care body will be described in more detail with reference to FIGS. 6 and 7.

FIG. 6 is a bottom view of a care body of a light output device illustrated in FIG. 1. FIG. 7 is a view for illustrating a care body of the present disclosure having an arrangement form of light sources based on a hair loss type.

Referring to FIGS. 6 and 7, a hair loss of a person 700 may mainly occur in a frontal area 711, frontotemporal areas 712 and 713, a parietal area 720, and/or a crown area 730.

Accordingly, according to the embodiment of the present disclosure, the plurality of light guide mechanisms 231 and the second laser light sources 245 that emit the laser light are arranged to correspond to the frontal area 711, the frontotemporal areas 712 and 713, the parietal area 720, and the crown area 730, thereby providing a more intensive care function for the areas.

On the other hand, because the hair loss is generally relatively less likely to occur in temporal areas, the light guide mechanisms 231 and the second laser light sources 245 may not be positioned at locations corresponding to the temporal areas, thereby providing an efficient care function, but the present disclosure may not be limited thereto.

As described above in FIG. 5, each of the plurality of light guide mechanisms 231 may be exposed to a bottom surface of the care body 2 via the openings 243 or the gap areas 244 of the substrate 241, thereby irradiating the laser light onto the user's scalp. In addition, light guide mechanism openings 252 corresponding to the plurality of light guide mechanisms 231 may be defined in the inner casing 25, thereby preventing an intensity of the laser light emitted from the light guide mechanisms 231 from decreasing when the laser light passes through the inner casing 25.

In particular, the light guide mechanisms 231 are implemented to distribute the laser light emitted from the first laser light sources 1000 and irradiate the laser light to a plurality of areas, and a spacing between the plurality of areas is smaller than a spacing between the second laser light sources 245. Accordingly, the light guide mechanisms 231 may irradiate the laser light more densely to a specific area, thereby maximizing the care effect. In addition, the light guide mechanisms 231 may irradiate the laser light to wide areas with one first laser light source 1000, thereby maximizing efficiency. The light guide mechanisms 231 will be described in more detail later with reference to FIGS. 8 to 12.

In one example, the light emitted from the second laser light sources 245 and the LEDs 246 may be irradiated to the user's scalp through the inner casing 25.

The plurality of LEDs 246 may be arranged in an evenly distributed manner in various areas of the substrate 241 to provide an overall care function for the various areas of the user's head.

Common hair loss types include M-type hair loss in which the hair loss progresses gradually from the frontotemporal areas 712 and 713, V-type hair loss in which the hair loss progresses gradually from the crown area 730, F-type hair loss in which the hair loss progresses gradually from the parietal area 720, U-type hair loss in which the hair loss progresses complexly in the frontal area 711, the frontotemporal areas 712 and 713, the parietal area 720, and the crown area 730, and the like.

In other words, an area requiring intensive care may vary depending on the user's hair loss type.

Accordingly, the light output device 1 according to the embodiment of the present disclosure may divide the laser light sources 1000 and 245 and the LEDs 246 disposed in the care body 2 into a plurality of zones (e.g., a ZONE1, a ZONE2, and a ZONE3).

The light output device 1 may sense the hair loss type of the user using the at least one image sensor 262 and the photodiodes 264, and control laser light sources 1000 and 245 and/or LEDs 246 included in at least one zone based on the sensed hair loss type.

Although not shown, an image sensor (not shown) for sensing a hair loss condition of the frontal area of the user may be further disposed on the inner surface of the outer casing 21.

For example, the image sensor (not shown) may be disposed at a front side of the inner surface of the outer casing 21 so as to face the frontal area of the user when the device is worn.

In this case, the light output device 1 may sense the user's hair loss type using the image sensor 262 disposed to capture images of the parietal area 720 and the crown area 730, and the image sensor disposed on the inner surface of the outer casing 21 to capture an image of the frontal area.

For example, a controller 550 (see FIG. 11) of the light output device 1 may turn on only laser light sources corresponding to the first zone ZONE1 and not turn on laser light sources corresponding to the second zone (ZONE2) and the third zone (ZONE3) when the sensed hair loss type is the M-type hair loss. Similarly, the controller 550 may turn on only LEDs corresponding to the first zone ZONE1 and not turn on LEDs corresponding to the remaining zones. However, depending on the embodiment, to provide the overall care function for the entire scalp, the controller 550 may turn on all the LEDs regardless of the hair loss type.

In one example, the light sources 1000, 245, and 246 may emit heat along with light as they emit light. In this case, a performance of the light sources 1000, 245, and 246 may gradually deteriorate by the heat.

In addition, the heat may cause sweat to be generated from the user's scalp, and the light emitted from the light sources 1000, 245, and 246 may be reflected by the sweat generated on the scalp, thereby deteriorating the care effect.

While controlling the light sources 1000, 245, and 246 to irradiate light onto the scalp, the controller 550 may obtain temperature and humidity information via the temperature/humidity sensor 266, and control the light sources 1000, 245, and 246 based on the obtained temperature and humidity information.

For example, when the obtained temperature or humidity is equal to or higher than a reference temperature or a reference humidity, the controller 550 may stop the light output of the light sources 1000, 245, and 246. The controller 550 may resume the light output of the light sources 1000, 245, and 246 after stopping the light output for a predetermined time, or may resume the light output when the temperature or the humidity obtained during the stop of the light output drops below a predetermined temperature or a predetermined humidity. Accordingly, the light output device 1 may perform the efficient care operation based on the temperature and the humidity.

Hereinafter, embodiments of a thin film-type heat dissipation sheet will be described in more detail with reference to FIGS. 8 to 10.

FIG. 8 is a view for illustrating a location of a thin film-type heat dissipation sheet 3 according to an embodiment of the present disclosure.

Referring to FIG. 8, the controller or a main board 551 that performs a role of the controller may be disposed at a rear side of the inner surface of the outer casing 21. Because the main board 551 performs a key role of controlling operations of the light sources, the sensors, and the various components of the light output device 1 for the scalp care and processing data, it is preferable that the main board 551 is located at the rear side to optimize a wiring length of the device and maximize a use of an internal space.

The main board 551 may control the plurality of light sources and the sensors of the light output device 1 for the scalp care, process an input command of the user, and manage an operating state of the device. The controller, which performs the role of the main board, will be described later with reference to FIG. 11.

In this regard, because the main board may include a light source operating circuit, a sensor control circuit, and a power management circuit, there is a risk of causing overheating and user inconvenience resulted from heat accumulation.

In one example, the thin film-type heat dissipation sheet 3 may be disposed at a location covering the main board 551, that is, in a space between the main board 551 and the inner casing 25. This is to prevent heat generated from the main board 551 from being transferred to the user's skin via the inner casing 25.

In addition, by blocking or diffusing heat from a heating element to uniformize the temperature within the skin care device, deterioration of the functions of the components disposed within the device may be prevented.

In this regard, the thin film-type heat dissipation sheet 3 may be formed with an area size equal to or greater than an area size of the main board 551, thereby effectively blocking the heat generated from the main board 551. In addition, the thin film-type heat dissipation sheet 3 needs to be thin and flexible so as be disposed in a narrow space of the device for the scalp care. The thin film-type heat dissipation sheet 3 may have a configuration that efficiently manages heat while having a small thickness.

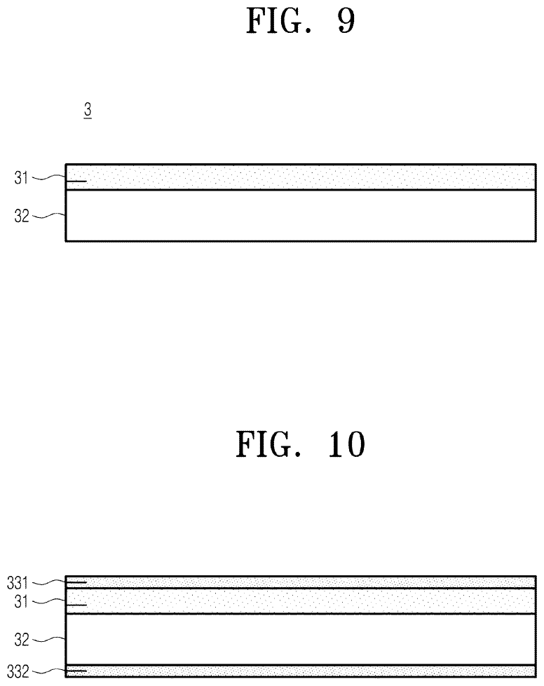

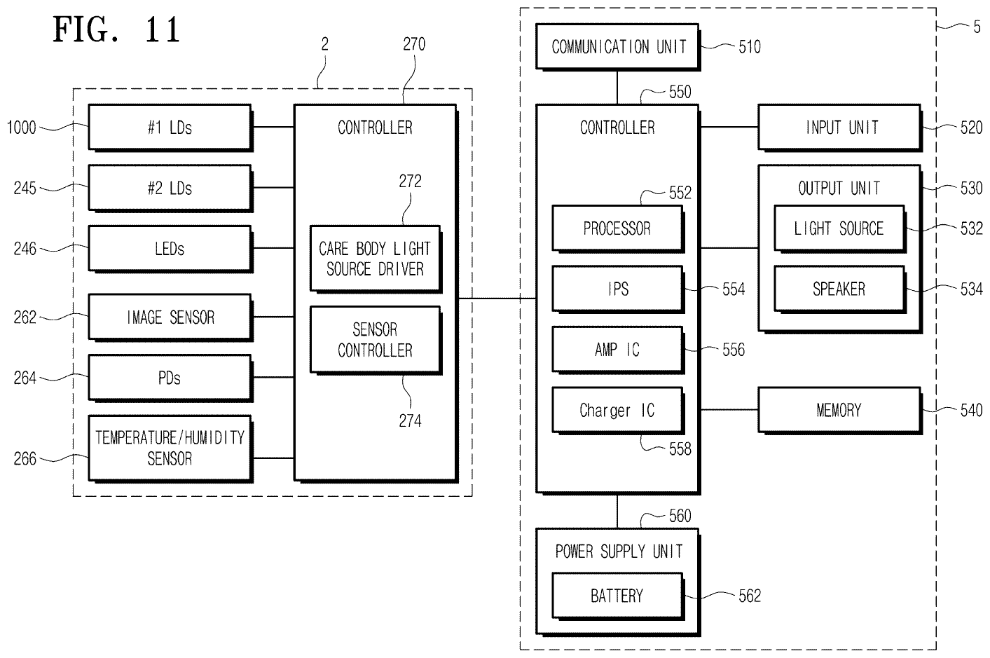

Hereinafter, referring to FIGS. 9 and 10, the configuration of the thin film-type heat dissipation sheet 3 will be described in detail.

FIG. 9 is a cross-sectional schematic diagram of a thin film-type heat dissipation sheet according to an embodiment of the present disclosure. FIG. 10 is a cross-sectional schematic diagram of a thin film-type heat dissipation sheet according to another embodiment of the present disclosure.

The thin film-type heat dissipation sheet according to an embodiment of the present disclosure may include an insulating layer 32, a heat diffusion layer 31, and a coating layer 331 and 332. The insulating layer 32 may block heat, and the heat diffusion layer 31 may disperse heat vertically and horizontally. The coating layer 331 and 332 may protect the insulating layer 32 and the heat diffusion layer 31 to reduce generation of dust and increase durability of the heat dissipation sheet.

A thickness of the insulating layer 32 is preferably no more than about 0.5 mm. To provide a high insulating effect while maintaining such small thickness, the insulating layer may be formed as a layer of a flexible material including an aerogel. The insulating sheet made of the aerogel has flexibility because of characteristics of the material, so that design thereof is easily changeable, the thickness thereof is adjustable, and insulating performance thereof is excellent.

Table 1 shows thermal conductivity of each material. Insulating materials are categorized into inorganic, organic, and high-performance insulating materials, and thermal conductivity and thickness of each are presented differentially.

The inorganic insulating material includes glass wool and rock wool, which are based on glass raw materials and silicate/calcium-based ores, respectively. They are relatively thick with thicknesses of 160 and 170 mm, and have thermal conductivity of 0.034 and 0.035 W/m·K, respectively, which shows average insulating performance.

The organic insulating material includes expanded polystyrene (EPS), extruded polystyrene (XPS), and polyurethane. The EPS and the XPS have relatively excellent insulating properties of 0.036 W/m·K at 145 mm thickness and of 0.027 W/m·K at 150 mm thickness, respectively. The polyurethane shows thermal conductivity of 0.023 W/m·K even at a small thickness of 110 mm, and thus is evaluated as the most efficient material among the organic insulating materials.

The high-performance insulating material includes an aerogel sheet and a vacuum insulating material. The aerogel sheet is composed of a combination of nonwoven fabric and aerogel, and shows thermal conductivity of 0.015 W/m·K even at a thickness of only 0.5 mm. The vacuum insulator is based on a silica core, shows very low thermal conductivity of 0.002 W/m·K at a thickness equal to or greater than 1.0 mm, but has insufficient structural flexibility, so that application in a narrow space may be limited.

TABLE 1

| | | | | Thermal |
|---|---|---|---|---|
| | | | Thickness | conductivity |
| Division | Material | | (mm) | (W/m · K) |
| Inorganic | Glass wool | Glass raw | 160 | 0.034 |
| | | material | | |
| | Rock wool | Silicate/Calcium- | 170 | 0.035 |
| | | based ore | | |
| Organic | EPS¹⁾ | Polystyrene | 145 | 0.036 |
| | XPS²⁾ | Polystyrene | 150 | 0.027 |
| | Polyurethane | Polyol + | 110 | 0.023 |
| | | Isocyanate | | |
| High- | Aerogel | Nonwoven | 0.5 | 0.015 |
| performance | sheet | fabric + | | |
| insulating | | aerogel | | |
| material | Vacuum | Silica core | >1.0 | 0.002 |
| | insulating | | | |
| | material | | | |

The insulating layer according to an embodiment of the present disclosure adopts the aerogel sheet in consideration of such characteristics. The aerogel sheet may provide excellent heat blocking performance and an ultra-thin structure at the same time. The thermal conductivity thereof is 0.015 W/m·K, which greatly surpasses those of the inorganic and organic insulating materials, and the thickness thereof is only 0.5 mm, so that it may be effectively applied even in the limited internal space. In addition, because of the flexible structure thereof combined with the nonwoven fabric, it is suitable for various device designs including curved surfaces, and is also able to contribute to a weight reduction of the device.

More specifically, the insulating layer 32 may be made of a composite material composed of glass fibers and silica gel particles. Such structure may provide the excellent heat blocking performance and structural stability by combining physical and chemical properties of the respective components.

The glass fibers may have high thermal resistance and mechanical strength, and may effectively block heat transfer within the insulating layer because of a fine structure thereof. In addition, fiber strength may be increased using a fabric structure.

The silica gel particles are nanometer-sized particles and maximize the heat blocking effect with a high specific surface area. A diameter of the silica gel particle may preferably be in a range of about 2 to 50 nm. This may be an ideal size that may maximize the insulating performance while maintaining the thickness of the insulating layer small.

The glass fibers and the silica gel particles are blended at an optimal ratio, which may approximately be a ratio of about 7 to 3 of the glass fibers and the silica gel. When such ratio is applied, an amount of dust shattering may be reduced compared to that in the existing case.

As such, the structural strength of the glass fibers and the excellent insulating performance of the silica gel may be maintained in a balanced manner.

In addition, manufacturing with an appropriate thickness may prevent occurrence of a tearing phenomenon.

Such composite structure forms a microporous structure, contributing to lowering the thermal conductivity, and simultaneously achieving lightweight and flexibility of the insulating layer.

In one example, the heat diffusion layer 31 may serve to disperse heat vertically or horizontally. Referring to FIG. 9, the heat diffusion layer 31 may be disposed on one surface of the insulating layer 32, and a layer disposed closer to the main board 551 may be either the heat diffusion layer 31 or the insulating layer 32. That is, a type of the layer that directly faces the main board does not significantly affect the thermal management effect of the heat dissipation sheet.

In addition, although not shown in the drawing, according to another embodiment of the present disclosure, the heat diffusion layers may be formed on both surfaces of the insulating layer. When the heat diffusion layers are formed on both surfaces, the heat diffusion effect may be further enhanced. In such case, each layer may be manufactured thinner, so that a total thickness of the layers does not exceed 1 mm.

In this regard, the heat diffusion layer 31 may be formed with expanded graphite as a main component, and may be composed of the expanded graphite alone or a combination of the expanded graphite and an additive such as metal particles and carbon nanotubes.

In particular, the expanded graphite is a material that has both high thermal conductivity and low specific gravity at the same time. Because of characteristics of the expanded graphite, heat may be quickly transferred in a thickness (vertical) direction where a heat transfer path is short, and heat may be evenly distributed in a surface (horizontal) direction where the heat transfer path is long.

Such dual heat transfer characteristics may effectively alleviate local heat concentration (hot spot) that may occur inside the device and contribute to maintaining overall heat distribution evenly.

In addition, it is desirable that the thickness of the heat diffusion layer is equal to or lower than approximately 0.4 mm. This is to optimize the function of the heat diffusion layer even in the limited internal space, while maintaining the weight reduction and the structural stability of the device at the same time. The thin heat diffusion layer based on the expanded graphite may sufficiently exhibit the thermal management performance even in a precise structure such as the light output device.

Table 2 shows results of comparing temperature changes and effects when applying the insulating material and a heat diffusion material between the main board and the inner casing. The table includes inner surface temperatures of a central portion of the main board and degrees of improvement for following cases: when no insulating material is applied, when only the insulating material (the aerogel sheet) is applied, and when the insulating material with an additional thermal diffusion material (a graphite sheet) is applied.

When no insulating material was applied, the inner surface temperature of the central portion of the main board was measured to be 51.6° C., which is a reference value. However, when the insulating material (the aerogel sheet) was applied, the inner surface temperature decreased by 3.2° C. to 48.4° C., which proves the excellent insulating performance of the aerogel. Furthermore, when the thermal diffusion material (the graphite sheet) was additionally applied together with the insulating material, the temperature decreased to 44.0° C., showing an additional temperature improvement effect of 4.4° C. and a total temperature improvement effect of 7.6° C. in temperature compared to the case in which the insulating material was applied alone.

In conclusion, applying the insulating material between the main board and the inner casing may reduce the inner surface temperature by 3.2° C., and additionally applying the thermal diffusion material may achieve the temperature improvement by up to 7.6° C.

TABLE 2

| | Control group | | Insulating material + |
|---|---|---|---|
| | no insulating | Insulating | thermal diffusion |
| Division | material applied | material | material |
| Material | — | Aerogel | Aerogel sheet + |
| | | sheet | graphite sheet |
| Thickness | — | 0.45 mm | 0.7 mm |
| Inner surface of | 51.6 | 48.4 | 44 |
| central portion of | | | |
| main board | | | |
| Comparison value | 0 | −3.2 | −7.6 |
| with control group | | | |

This shows that the combination of the aerogel and the expanded graphite may exhibit very effective performance in the heat management. The composite material based on the aerogel and the expanded graphite according to an embodiment of the present disclosure may simultaneously satisfy the insulation and heat diffusion performance, thereby improving stability and efficiency of the device. Such results may support technical potential of the present disclosure and may contribute to meeting various heat management requirements of the skin care device, a display, or the like.

Referring to FIG. 10, the thin film-type heat dissipation sheet 3 may further include the coating layer 331 and 332 attached to the other surface of the insulating layer 32 or one surface of the heat diffusion layer 31. The coating layer may be formed on one surface or both surfaces of the composite layer including the insulating layer and the heat diffusion layer.

In addition, although not shown in the drawing, the coating layer may also be formed on side surfaces of the insulating layer and the heat diffusion layer so as to completely enclose the composite layer. In such a case, by completely coating all surfaces, fine dust that may be generated from the insulating layer and the heat diffusion layer may be prevented from shattering. In addition, the coating layers 331 and 332 may protect each layer of the heat dissipation sheet from the outside and improve the durability.

The coating layer may be based on a polyethylene terephthalate (PET) film, and an adhesive layer made of an acrylic, silicone, or urethane-based material may be attached onto the film. In addition, flame retardancy is secured using a PI film, so that heat reduction is available.

A thickness of the adhesive layer may be preferably in a range of 0.05 mm to 0.15 mm, and a thickness of the PET film may be in a range of 0.2 mm to 0.5 mm. The adhesive layer helps the coating layer to be stably fixed, and a thickness of the coating layer itself is preferably set to be in a range of about 0.05 mm to 0.15 mm.

In this regard, the coating layer may be manufactured in a scheme of manufacturing the aerogel sheet and then laminating an entire surface of the sheet using a flame retardant film.

FIG. 11 is a block diagram showing control components of a light output device for scalp care according to an embodiment of the present disclosure.

In FIG. 11, for convenience of description, it is assumed that the control components of the light output device 1 are distributed in the care body 2 and the user manipulation device 5, but all of the control components may be equipped in the care body 2 depending on the embodiment.

When all of the control components are equipped in the care body according to an embodiment of the present disclosure, as described above, the control components may become a heat source, and thus the heat dissipation sheet for alleviating the heat emitted from the heat source may be required.

In this regard, the heat dissipation sheet is located in the very narrow space, so that the heat dissipation sheet should be structured to be very thin while efficiently blocking heat.

Referring again to FIG. 11, the care body 2 of the light output device 1 may include the plurality of first laser light sources 1000, the plurality of second laser light sources 245, the plurality of LEDs 246, the at least one image sensor 262, the temperature/humidity sensor 266, and a controller 270.

The plurality of first laser light sources 1000 and the second laser light sources 245 may be implemented as the laser diodes that emit the laser light. As described above, a light amount (the output) of the first laser light source 1000 may be greater than a light amount of the second laser light source 245. Further, the number of first laser light sources 1000 may be smaller than the number of second laser light sources 245.

The plurality of first laser light sources 1000 may be arranged to correspond to the plurality of light guide mechanisms 800 as described above in FIGS. 8 to 12. The plurality of first laser light sources 1000 may not be positioned to directly face the scalp when the device is worn.

The plurality of second laser light sources 245 may be mounted on the care body light source mounting portion 24 to be spaced apart from each other as illustrated in FIGS. 5 to 7.

In one example, the first laser light sources 1000 and the second laser light sources 245 may be arranged to correspond to the areas including the frontal area 711, the frontotemporal areas 712 and 713, the parietal area 720, and the crown area 730 of the user as described above in FIG. 7.

The plurality of LEDs 246 may be arranged in the various areas of the substrate 241 to irradiate light to the various areas of the user's scalp.

In one example, each of the first laser light sources 1000, the second laser light sources 245, and the plurality of LEDs 246 may emit the red light having the wavelength of about 630 nm to 670 nm. The red light may promote the hair growth by promoting the activation of the hair follicles.

The at least one image sensor 262 may obtain an image including the user's head area. The controller 550 may sense the hair loss condition, the hair loss type, and the like of the user based on the obtained image. Depending on the embodiment, each of the second laser light sources 245 may be equipped with the photodiode 264. In this case, the controller 550 may sense the hair loss condition, the hair loss type, and the like using the at least one image sensor 262 and the photodiode 264.

The temperature/humidity sensor 266 may sense a temperature and a humidity of an area adjacent to the user's scalp during the operation of the light output device 1. The controller 550 may control the light output of the light sources 1000, 245, and 246 based on the sensed temperature and humidity.

The controller 270 equipped in the care body 2 may include a care body light source driver 272 that controls on/off of the light sources 1000, 245, and 246, and a sensor controller 274 that controls the operation of the sensors 262, 264, and 266.

When a control signal for each of the light sources 1000, 245, and 246 is received from a processor 552 of the controller 550, the care body light source driver 272 may control the light output of each of the light sources 1000, 245, and 246 based on the received control signal.

When a control signal for each of the sensors 262, 264, and 266 is received from the processor 552, the sensor controller 274 may also control the operation of the sensors 262, 264, and 266 based on the received control signal. The sensor controller 274 may transmit sensing data received from each of the sensors 262, 264, and 266 to the processor 552.

Depending on the embodiment, the care body light source driver 272 and the sensor controller 274 may be implemented within the controller 550 or may be implemented integrally with the processor 552.

In one example, the user manipulation device 5 of the light output device 1 may include a communication unit 510, an input unit 520, an output unit 530, a memory 540, the controller 550, and a power supply unit 560.

The communication unit 510 may include at least one communication module for connecting the light output device 1 to a user's mobile terminal (a smartphone, a tablet PC, and the like), a server, or the like. For example, the at least one communication module may support a short-range wireless communication scheme such as Bluetooth or may support a wireless Internet scheme such as Wi-Fi.

For example, the controller 550 may transmit operation or state information of the light output device 1 to the user's mobile terminal via the communication unit 510. In addition, the controller 550 may transmit scalp condition information, hair loss condition information, and/or hair loss type information of the user to the user's mobile terminal via the communication unit 510. The scalp condition information, the hair loss condition information, and the hair loss type information may be information obtained based on the sensing data of the image sensor 262 and/or the plurality of photodiodes 264.

The input unit 520 may receive input related to power on/off of the light output device 1, setting of the operation mode, and the like from the user. For example, the input unit 520 may include at least one button.

The output unit 530 may output information such as a power state, an operation mode, and a battery state of the light output device 1. For example, the output unit 530 may include at least one light source 532 and a speaker 534 that outputs the information in a form of sound.

The memory 540 may include control data for controlling the components included in the light output device 1 or data related to light output settings of the light sources 1000, 245, and 246 based on each of the plurality of operation modes.

In addition, the memory 540 may include data or an algorithm for generating the scalp condition information, the hair loss condition information, and/or the hair loss type information from sensing values provided from the image sensor 262 and/or the plurality of photodiodes 264.

In addition, the memory 540 may include data or an algorithm for controlling the light output of the light sources 1000, 245, and 246 based on the temperature and humidity information provided from the temperature/humidity sensor 266.

The memory 540 may be understood as a concept encompassing at least one volatile memory (a RAM or the like) and at least one non-volatile memory (a ROM, a flash memory, or the like).

The controller 550 may control the overall operation of the light output device 1. Such controller 550 may include at least one processor (or controller). In addition, the controller 550 may include at least one CPU, an application processor (AP), a microcomputer, an IC, an application specific integrated circuit (ASIC), and the like in terms of hardware.

For example, the controller 550 may include the processor (main processor) 552, an image signal processor (ISP) 554, an amplifier IC 556, a charging IC 558, and the like.

The processor 552 may correspond to the main processor that controls the overall operation of the light output device 1. For example, the processor 552 may set the operation mode of the light output device 1 based on the input received via the input unit 520, and control the components included in the light output device 1 based on the set operation mode. In addition, the processor 552 may control the operations of other components 554, 556, and 558 included in the controller 550, and also the operations of the care body light source driver 272 and the sensor controller 274 of the care body 2.

In one example, the processor 552 may sense the hair loss condition or the hair loss type of the user based on the sensing values obtained from the image sensor 262 and/or the plurality of photodiodes 264. The processor 552 may control the light output of the light sources 1000, 245, and 246 corresponding to the at least one zone among the plurality of zones ZONE1 to ZONE3 (see FIG. 7) based on the sensed hair loss condition or hair loss type.

The ISP 554 may process the sensing value obtained from the image sensor 262 to generate the image. The generated image may include the user's scalp. The processor 552 may transmit the generated image to the user's terminal or the like via the communication unit 510.

The amplifier IC 556 may control the sound output of the speaker 534 included in the output unit 530, and the charging IC 558 may control charging or power supply of a battery 562 of the power supply unit 560.

The power supply unit 560 may provide power required for the operation of the light output device 1 to each of the components. For example, the power supply unit 560 may include the battery 562. The power supply unit 560 may include a terminal for connecting to an external power supply source, and may charge the battery 562 with power supplied from the outside via the terminal. The power supply unit 560 may supply the power to the components included in the care body 2 via the cable 52.

As described above, the present disclosure provides the configuration that may effectively improve the heat management performance via the combination of the insulating material and the heat diffusion material. The aerogel-based insulating layer exhibits the excellent heat blocking effect, and the expanded graphite-based heat diffusion layer helps maintain the temperature inside the device evenly by dispersing the concentrated heat. This prevents excessive temperature rise resulted from the heat of the device, protecting the user's skin and providing a safe environment even in a case of long-term use.

In addition, the heat management may contribute to increasing the durability of the device by minimizing damage related to the overheating of the internal components. The composite material of the present disclosure, which may sufficiently exhibit the insulation and heat diffusion performance even in the limited space, may be suitably used in the various environments requiring the heat management, and may play an important role in improving the performance of the device such as the light output device for the scalp care and a user experience.

It will be apparent to those skilled in the art that the present disclosure may be embodied in other specific forms without departing from the spirit and essential characteristics of the present disclosure.

The detailed description above should not be construed in any way as restrictive but rather as illustrative. The scope of the present disclosure should be determined by a reasonable interpretation of the appended claims, and all changes within the equivalency range of the present disclosure are included within the scope of the present disclosure.

What is claimed is:

1. A light output device for scalp care including a thin film-type heat dissipation sheet, the device comprising:
a dome-shaped outer casing forming an outer appearance of the device;
an inner casing formed inside the outer casing;
a plurality of light sources arranged in a space between the outer casing and the inner casing;
a controller disposed in the space between the outer casing and the inner casing; and
the thin film-type heat dissipation sheet positioned adjacent to the controller,
wherein the thin film-type heat dissipation sheet includes:
an insulating layer made of a flexible material including an aerogel; and
a heat diffusion layer formed on one surface of the insulating layer.

2. The device of claim 1, wherein the controller is disposed in a rear side of the space between the outer casing and the inner casing,
wherein the thin film-type heat dissipation sheet is disposed in a space between the controller and the inner casing.

3. The device of claim 1, wherein the thin film-type heat dissipation sheet further includes a coating layer attached to a remaining surface of the insulating layer or one surface of the heat diffusion layer.

4. The device of claim 3, wherein the coating layer is formed on all of the remaining surface of the insulating layer, the one surface of the heat diffusion layer, a side surface of the insulating layer, and a side surface of the heat diffusion layer to surround the insulating layer and the heat diffusion layer.

5. The device of claim 1, wherein the heat diffusion layer is formed on both surfaces of the insulating layer.

6. The device of claim 1, wherein the insulating layer includes glass fibers and silica gel particles having a diameter in a range of 2 to 50 nm.

7. The device of claim 6, wherein a ratio of the glass fibers and the silica gel particles is substantially 7 to 3.

8. The device of claim 1, wherein the heat diffusion layer is made of expanded graphite.

9. The device of claim 1, wherein a thickness of the insulating layer is equal to or smaller than 0.5 mm.

10. The device of claim 1, wherein a thickness of the insulating layer is equal to or smaller than 0.4 mm.

11. The device of claim 1, wherein the thin film-type heat dissipation sheet has a total cross-sectional thickness equal to or smaller than 1 mm.
